(19) United States
(12) Patent Application Publication
(10) Pub. No.: US 2014/0199266 A1
(43) Pub. Date: Jul. 17, 2014

(54) HONEY NASAL RINSE

(71) Applicant: LOST CREEK CONSULTING, LLC, Springville, UT (US)

(72) Inventors: Roland Clifford Park, Springville, UT (US); Kenneth Gene Rayl, Kaysville, UT (US); Ian Joseph Alexander, Albuquerque, NM (US)

(73) Assignee: LOST CREEK CONSULTING, LLC, Springville, UT (US)

(21) Appl. No.: 14/155,064

(22) Filed: Jan. 14, 2014

Related U.S. Application Data
(60) Provisional application No. 61/752,053, filed on Jan. 14, 2013.

(51) Int. Cl. A61K 35/64; A61K 9/00; A61K 45/06

(52) U.S. Cl. CPC A61K 35/644; A61K 45/06; A61K 9/0043
USPC 424/93.1; 424/537

(57) ABSTRACT

Novel nasal rinse compositions are disclosed. The nasal rinse comprises a dried particulate composed of honey and a carrier. The composition may be a co-processed composition wherein the honey and carrier are combined while the honey is in liquid form and processed together to produce the dried particulate. Methods for producing the nasal rinse compositions are also provided, as well as methods of use of the nasal rinse compositions for the prevention or treatment of nasal infection, rhinitis, or sinusitis.

### CROSS-REFERENCE TO RELATED APPLICATIONS

This application claims priority under 35 U.S.C. §119 to provisional application Ser. No. 61/752,053 filed Jan. 14, 2013, herein incorporated by reference in its entirety.

### FIELD OF THE INVENTION

The present invention relates generally to compositions and methods for nasal rinses composed of honey and a carrier. The nasal rinse compositions include nasal rinse powders that comprise a dried particulate composed of honey and a carrier. The composition may be a co-processed composition wherein the honey and carrier are combined while the honey is in liquid form and processed together to produce the dried particulate.

### BACKGROUND OF THE INVENTION

A sinus is a hollow space within the bones of the face. Humans have several sinuses. The sinuses humidify and warm the inhaled air, add to the sense of smell and taste, and contribute to speech abilities. The sinuses and the nasal passage that runs from the nostrils to the pharynx are lined with mucosa. Sinusitis is an inflammation of the mucosa of the sinuses and rhinitis is an inflammation of the mucosa of a nasal passage.

Sinusitis and rhinitis may be caused by cold viruses, allergies to various allergens, smoking, bacterial or fungal infections, nasal polyps, deviated nasal septums, and non-allergic hypersensitivities. Symptoms of rhinitis may include: stuffy nose, runny or drippy nose, scratchy throat and dry cough. Symptoms of sinusitis may be severe and may occur when the sinuses are inflamed and ostia are blocked. Symptoms may include: nasal congestion, runny or stuffy nose, white, yellow or green discharge, headache, night time cough, pain in the upper jaw or teeth, persistent fatigue, fever, loss of sense of smell or taste, and sometimes serious infections like meningitis, brain abscess, or ear infections.

Current treatments for relief of sinusitis, rhinitis, nasal congestion, or treating the symptoms of sinus disease include utilizing a nasal rinse to reduce or permanently cure the symptoms. Currently available nasal rinses include a variety of saline or water based nasal rinses, or prescription and over-the-counter drug allergy medicines. However, these current treatments may be ineffective for severe congestion, may not treat allergy issues, may not relieve related symptoms and may require a prescription for utilization. In addition, travel restrictions, space availability, and expiration limitations may affect the utilization of nasal rinses.

### SUMMARY OF THE INVENTION

In one aspect, the invention includes compositions and methods for effective treatment of nasal diseases or conditions.

In another aspect, the invention includes liquid or powder nasal rinse compositions with enhanced anti-bacterial properties.

In another aspect, the invention includes liquid or powder nasal rinse compositions comprising honey.

In another aspect, the invention includes methods for preparing co-processed compositions, including nasal rinse compositions.

In another aspect, the invention includes co-processed compositions, comprising a dried particulate comprising honey and a carrier.

In another aspect, the invention includes systems for preparing a composition for the treatment or prevention of a nasal infection, rhinitis, or sinusitis.

In another aspect, the invention includes methods for treating or preventing a nasal infection, rhinitis, or sinusitis, comprising administering a nasal rinse solution.

One embodiment of the present invention provides a co-processed composition. The co-processed composition may include a dried particulate of a carrier and honey in which the ratio of the carrier to honey is 99:1 to 1:99.

Another embodiment provides a nasal rinse powder. The nasal rinse powder may include a dried honey particulate. The nasal rinse powder may also include a salt based constituent. The nasal rinse powder may include dried honey bound to a salt carrier and an additional carrier.

Yet another embodiment includes a method for manufacturing a powdered nasal rinse. In a preferred embodiment, honey in liquid form is combined with a first carrier to form a carrier mixture, and the carrier mixture is dried to form a powdered carrier mixture. The powdered carrier mixture is mixed with a second carrier to form the powered nasal rinse.

Yet another embodiment provides a method for manufacturing a powdered nasal rinse. Honey in liquid form may be combined with one or more carriers to form a carrier mixture. The carrier mixture may be dried to form the powdered nasal rinse.

### BRIEF DESCRIPTION OF THE DRAWINGS

Illustrative embodiments of the present invention are described in detail below with reference to the attached drawing figures, which are incorporated by reference herein and wherein:

FIG. 1 shows a pictorial representation of containers for storing or utilizing a nasal rinse in accordance with an illustrative embodiment.

FIG. 2 shows a flowchart of an exemplary co-processing procedure for creating a nasal rinse powder in accordance with an illustrative embodiment.

### DETAILED DESCRIPTION OF THE INVENTION

The illustrative embodiments provide a nasal rinse powder, nasal rinse solution, nasal rinse concentrate, nasal rinse gel, and method of manufacture and use. In one embodiment, the nasal rinse is packaged in any number of containers or application devices. The nasal rinse powder may be combined with distilled water, saline, or sterile water to create an aqueous solution for performing irrigation. The nasal rinse powder may include natural preservatives and may be pH balanced for maximum efficacy. The pH of the nasal rinse powder may be configured to be slightly acidic or slightly alkaline based on the needs of the user.

The various embodiments may also be utilized as a topical treatment, wound rinse, cleanser, or other health treatment as currently described or as combined with existing treatments. For example, the powder may be integrated into bandages, shampoos, facial cleansers, preservatives, antibiotic creams, and so forth. In another example, the powder may be used as a mouth or throat rinse, for gargling or similar treatment of the throat and/or the esophagus. The illustrative embodiments are much cheaper than synthetically created antibiotics. As a result, the various embodiments may be more easily tested and distributed to a number of users enhancing benefit to users from the illustrative embodiments.

As used herein, “honey” refers to generally to that produced by honey bees (Apis genus), especially A. mellifera. The honey of the present invention may also be produced by other species of honey bee, including Apis florea, Apis andreniformis, Apis dorsata, Apis dorsata binghami, and Apis dorsata laboriosa. As used herein, honey may also refer to biologically engineered or medicinal honeys.

In one embodiment, the nasal rinse powder includes natural or biologically engineered ingredients including honey. The honey may be in raw, processed, or powdered form. Honey is known to have anti-bacterial properties that may eliminate or reduce bacteria that cause sinus infections. Honey has been known to kill bacteria, such as Escherichia coli, Entrobacter cloacae, methicillin-resistant staphylococcus aureus (MRSA), Pseudomonas aeruginosa, Shigella dysenteriae, Klebsiella sp., Haemophilus influenzae, Proteus sp., Staphylococcus aureus, Streptococcus hemolyticus group B, Helicobacter pylori, and Candida albicans, including drug-resistant strains. (Noori S. Al-Waili. Journal of Medicinal Food. June 2004, 7(2): 210-222; Manisha Deb Mandal, Shyamapada Mandal, Asian Pac J Trop Biomed. 2011 April; 1(2): 154-160). Honey also has been demonstrated to possess antiviral properties. (Aamir Shahzad & Randall J Cohrs, Transl Biomed. 2012; 3(2): 2. doi: 10.3823/434). For example, honey may be effective in killing as much as 100% of planktonic and biofilm-grown bacteria. In addition, the bactericidal rates for the Sidr and Manuka honeys against MSSA, MRSA, and PA biofilms have been shown to be approximately 63-82 percent, 73-63 percent, and 91-91 percent, respectively. These rates are significantly higher (P<0.001) than those seen with single antibiotics commonly used against Staphylococcus aureus. The nasal rinse powder and solution may be produced utilizing honey that is associated with a particular region or location for reducing allergy symptoms associated with the region. For example, the honey may include small doses of pollens, spores, and allergens that may be utilized to decrease user sensitivity to those allergens. For example, regular application of the nasal rinse may help the user overcome allergies by lessening sensitivity. Not all honeys are of equal value, and in a preferred embodiment those with strong anti-bacterial properties are utilized. The honey utilized in the nasal rinse powder may be collected in regions, cities, localities, or other geographic areas to provide area-specific nasal rinse powders for customized treatment. The honey may be fresh raw honey, stored raw honey, heated honey, ultraviolet-exposed honey, genetically enhanced honey, medically treated honey, and heated stored honey, or otherwise processed honey.

In one embodiment, the protein defensin-1 was found to be part of bee's immune system and may have the antibiotic properties and effects herein described. Production of the defensin-1 protein may be naturally or synthetically enhanced in honey or the illustrative embodiments to provide additional medical benefits and properties.

In one embodiment, the honey may be Manuka (Leptospermum scoparium) honey from New Zealand which is known to have extremely strong anti-bacterial properties. The Sidr honey from Yemen may also have additional antimicrobial properties. The honey may also enhance the nasal rinse with a more enjoyable scent and taste. For example, medicinal-grade Manuka has high methyglycoxal (MGO or mogo) properties, and is reported to have other micronutrient agents that act to enhance its antibiotic activity and flavor. The honey may also be honey indigenous to the region where the product is being distributed as local honeys are known to have a greater level of efficacy in treatment of allergy related issues. For example, clover honey, Tulang (Koompassia excels) honey, Revamil, medihoney, and other natural, synthentic, or mixtures or compositions of honey or honey simulate/analog may be utilized.

In another embodiment, the honey may be a biologically engineered or processed honey. For example, a number of new biologically engineered honeys have been found to kill bacteria, include antibiotic properties, and foster wound and infection treatment. For example, the honey in the nasal rinse may be Surgihoney™

In one aspect, the nasal rinse powder may be packaged in stick packets, bags, bottles or other delivery containers for transfer, dilution, or aqueous or spray delivery. For example, the nasal rinse powder may be mixed with sterilized water in a neti-pot for irrigating the nasal passageways of a user. In another embodiment, the nasal rinse powder may be delivered in a slow release gel form (powder or liquid). The nasal rinse may also be delivered utilizing a pulse irrigator, such as a Water Pik or analogous device. In one embodiment, the nasal rinse may be encapsulated in fast dissolving pills, tablets, gelpacks, or packets that may be added to a container for irrigation.

Utilizing nasal rinsing allows the nasal rinse solution to be dispensed into the nasal passage to cleanse and wash away mucus and allergy creating particles and irritants. Nasal rinsing may allow the sinuses to drain normally and reduces the inflammation of the mucus membrane. In one aspect, the nasal rinse powder may be utilized for methods of treating sinusitis, rhinitis, nasal infection, including, for example, sinus symptoms, nasal allergies, cold and flu symptoms, nasal irritation, post nasal drip, congestion, and dryness. The nasal rinse powder, and solutions made therefrom, may have direct antimicrobial activity, or may stimulate increased host antimicrobial activity.

As used herein, the terms “treating”, “treat”, or “treatment” refer to: (i) preventing a disease, disorder or condition from occurring in a mammal, animal or human that may be predisposed to the disease, disorder and/or condition but has not yet been diagnosed as having it (e.g. prophylaxis); (ii) inhibiting the disease, disorder or condition, i.e., arresting its development; and/or (iii) relieving the disease, disorder or condition, i.e., causing regression of the disease, disorder and/or condition. In one aspect of the invention, for example, with respect to sinusitis, rhinitis, nasal infection, the compositions of the present invention may be administered in an effort to relieve symptoms such as stuffy nose, runny or drippy nose, scratchy throat and dry cough; to abrogate, kill or remove causative agents or conditions, such as cold viruses, allergies to various allergens, smoking, bacterial or fungal infections, nasal polyps, deviated nasal septums, and non-allergic hypersensitivities; or to prevent any of the proceeding from developing or occurring.

In the present invention, an “effective amount” or “therapeutically effective amount” of a compound or of a composition of the present invention is that amount of such compound and/or composition that is sufficient to affect beneficial or desired results as described herein. In terms of treatment of a mammal, e.g., a human patient, an “effective amount” is an amount sufficient to at least slow the progression or spread of disease, or render the disease susceptible to therapeutics or remediation.

Although the nasal rinse focuses on nasal irrigation, the described rinse may also be utilized for natural rinsing and relief of the mouth, throat (esophagus), ear cavities, other orifices, or as a topical rinse. In another embodiment, the nasal powder may be encapsulated in a slow release powder that may be dispersed in the nasal passages and slowly dissolve based on contact with tissue, membranes, water, and other bodily fluids to provide the benefits herein described. For example, the powder may be distributed in a dry form and then slowly dissolve into a liquid or gel based on contact with the user to provide the various benefits herein described. As a result, the nasal powder may be utilized as a medical treatment rather than a simple rinse.

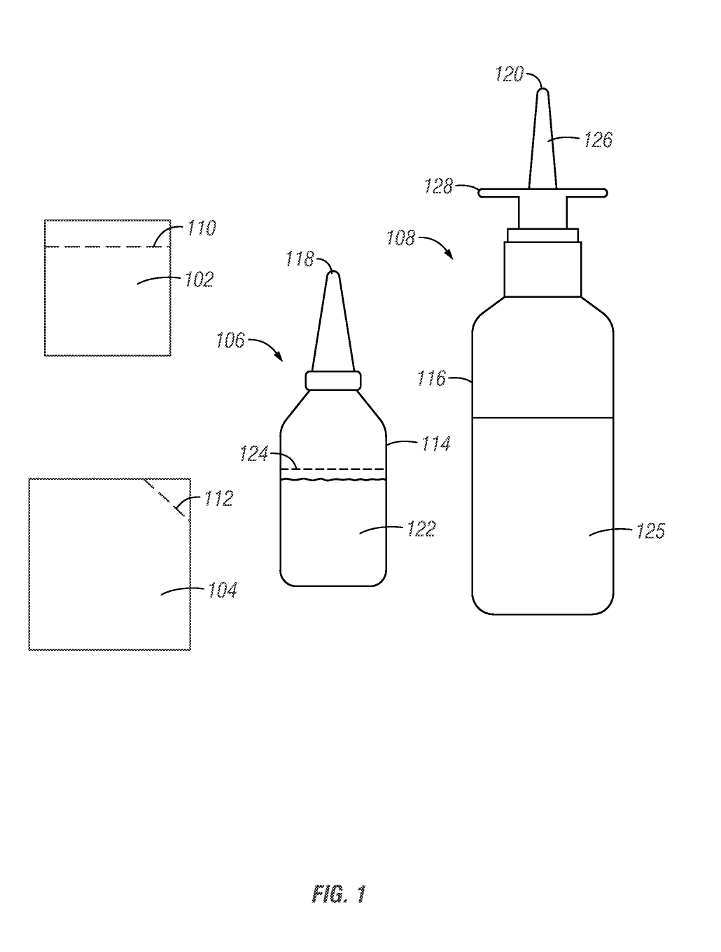

FIG. 1 shows a pictorial representation of containers for storing and utilizing a nasal rinse in accordance with an illustrative embodiment. The containers may include packets 102 and 104 of various sizes, rinse bottle 106, and spray bottle 108. In one embodiment, the nasal rinse powder may be inserted into packets in predetermined quantities. In other embodiments, the containers may include bulb syringes or other delivery containers as well as mechanical or electrical delivery systems, such as WaterPik. The rinse bottle 106 and the spray bottle 108 may be filled or manufactured with the nasal rinse powder, solution, gel, or concentrate. The various embodiments may also be applied to the sinuses of the user utilizing a swab, brush, powered sprayer, or so forth. In one embodiment, the nasal rinse powder may be created in a semi-aqueous state for mixture with additional water according to mixing instructions for the amount of concentrate.

The packets may be sized for a single irrigation or for multiple irrigation applications. For example, the packet 102 may be a single use rinse stick packet and the packet 104 may be utilized for multiple rinses or rinses requiring a greater volume. In other embodiments, the nasal rinse powder may be stored in bottles, bags, packages, vials, water soluble holders, or other convenient containers.

In one embodiment, the packet 102 is mixed with 4 ounces of distilled water, sterile water, a saline solution, a diluent, carrier, or other aqueous solution. However, the packet 102 and nasal rinse powder or solution may be effective when mixed or created to provide only one to two milliliters of nasal rinse. The amount of solution required to be used may be reduced because of the effectiveness of the nasal rinse (regardless of how it is produced, mixed, or so forth). In one embodiment, the packet 104 may be mixed with 8 ounces. The illustrative embodiments may also be utilized with customer delivery bottles or systems, such as a Nettie pot.

The packets 102 and 104 may include an aluminum lining or other protective lining or inner layer to protect the nasal rinse power from moisture, clumping, and oxidation. The packets 102 and 104 may include an indicator 110 and 112 that informs a user where to tear, cut, or open the packet. The indicators 110 and 112 may be a dotted line, printed or integrated line, perforation, resealable mechanism, or so forth. The indicators 110 and 112 may mark an exterior portion of the packets 102 and 104 that may be most convenient for cutting or otherwise opening the packet. In another example, the container may be a bottle that includes a cap sized to measure the nasal rinse volume in either a powder or liquid form.

The rinse bottle 106 and spray bottle 108 may be any number of delivery containers known in the art. For example, the rinse bottle 106 and the spray bottle 108 may have flexible sidewalls 114 and 116 and an opening 118 and 120 for a removable cap. The rinse bottle 106 and the spray bottle 108 may come pre-loaded with the nasal rinse or may be subsequently filled and mixed with the nasal rinse powder or concentrate.

In one embodiment, the rinse bottle 106 may also include nasal rinse 122 and a fill line 124. In one embodiment, the packet 102 may be added to the rinse bottle 106 and then water or another liquid may be utilized to fill the rinse bottle 106 to or approximate the fill line 124. In another embodiment, the rinse bottle 106 may include a number of fill lines that may be utilized with different amounts of nasal rinse powder. The rinse bottle 106 or spray bottle 108 may be utilized in a vertical configuration as shown, at an angle, inverted, or any combination thereof.

The spray bottle 108 may similarly include a spray top 126 and a pump 128 including internal pumping components (not shown). In one embodiment, the rinse bottle 106 or the spray bottle 120 may be pre-filled with a nasal rinse solution or powder before being packaged and sold to users. As a result, the nasal rinse powder or solution may be more easily utilized. The rinse bottle 106 and the spray bottle 120 may be utilized to mix, fill, or store the nasal rinse before or after being prepared. The size of the openings 118 and 120 may vary based on whether the nasal rinse is produced in a liquid or gel form.

In one embodiment, the spray bottle 108 may include a conduit in the cap's interior, which conduit extends into a bottom portion of the spray bottle when filled to utilize all of nasal rinse 125. For example, the pump of the spray bottle 108 may pump the nasal rinse from the bottom of the spray bottle 108 through the opening 120 for rinsing the nasal cavity of the user. The flexible sidewalls 114 and 116 of the rinse bottle 106 or spray bottle 108, when filled with the saline solution, are compressed to urge the nasal rinse through the openings 118 and 120 or through the conduit, or tube and conduit, and through the opening 118 and 120 in the cap and into a nasal passage. A top portion or cap of the rinse bottle 106 and the spray bottle 108 may be pressed against a nostril to provide a comfortable and effective seal.

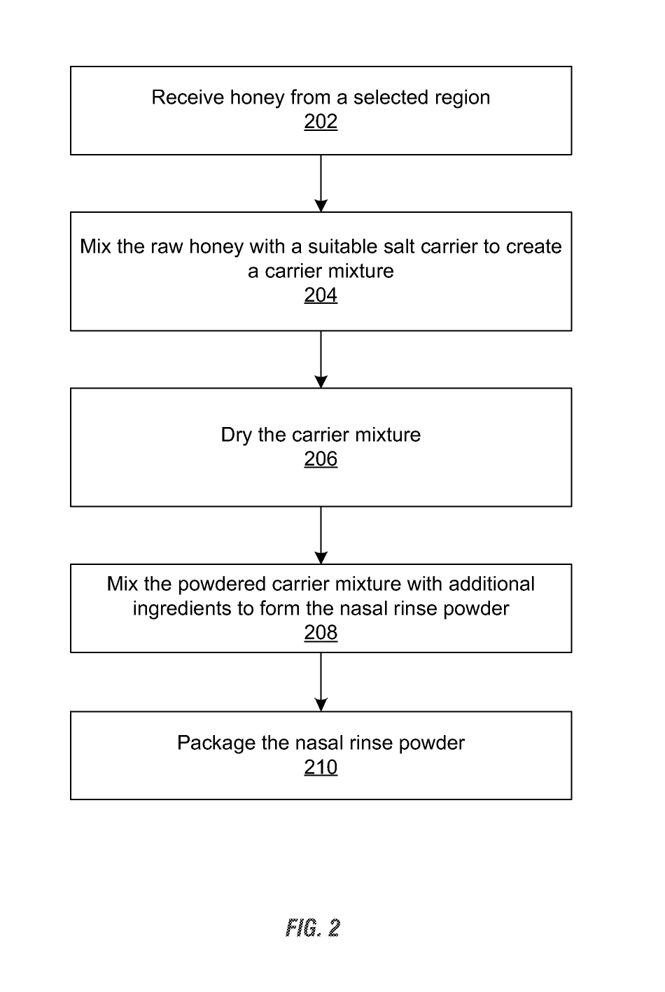

FIG. 2 is a flowchart of a process for creating a nasal rinse powder in accordance with an illustrative embodiment. The manufacturing process of FIG. 2 may be implemented by one or more devices, systems, or users as a process or method to create a nasal rinse powder, dried honey mixture, nasal rinse gel, nasal rinse concentrate, or so forth. The devices may include, but are not limited to, scales, solution mix tanks and mixers, pumps, heat transfer elements, dryers, screeners, blenders, packaging equipment, and so forth. The process may begin by receiving honey from a selected region (step 202). In one embodiment, the honey may be selected for generating batches for particular regions, countries, states, counties, or so forth. The nasal rinse powder may be manufactured for distribution in the selected regions.

Next, the system mixes the raw honey with a suitable salt carrier and water to create a carrier mixture (step 204). In one embodiment, sodium chlorite or sodium chloride is the carrier. However, at low temperatures and concentrations of honey, sodium bicarbonate may be a suitable carrier. The ideal carrier has high solubility in water, is solid at room temperature, is approximately neutral pH in water, is not hygroscopic, and is physically stable when dried. However, under certain process conditions it may be possible to use an alternative carrier which does not meet all the criteria listed above. Alternate carriers may include gums: gum arabic, guar gum, xanthan gum, tara gum, carrageenan and gelatin; mineral salts: calcium carbonate, sodium carbonate, sodium bicarbonate, potassium bicarbonate, magnesium carbonate, potassium chloride; vitamins: Vitamin A, Vitamin C, all B Vitamins, Vitamin B12 (e.g. Cyanocobalamin, hydroxocobalamin, etc.), Vitamin D, Vitamin E, Vitamin K, Zinc, niacin, thiamine, riboflavin, pyridoxine, etc.; cellulose and cellulose derivatives: microcrystalline cellulose, powdered cellulose, methylcellulose, ethylcellulose, hydroxypropyl cellulose, hydroxypropyl methylcellulose, carboxymethylcellulose; sugars and sugar alcohols: sucrose, fructose, dextrose, maltose, lactose, dextrin, mannitol, sorbitol, maltitol, xylitol; starch and starch derivatives: maltodextrin, cyclic oligosaccharides (cyclodextrins); amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine; soy products including soy protein. The raw honey may be mixed in its natural form with the carrier to ensure the carrier and honey become a co-processed physical matrix composition.

As used herein, “co-processing” or “co-process” encompasses combining two or more established compounds or components by an appropriate process. Co-processing may include further processes or procedures that physically mix and/or treat, prepare, or refine the composition of the two or more combined compounds or components, for example by blending, mixing, rolling, or other similar processes. The co-processing may also include subsequent drying processes to remove water from the composition, Co-processing of compounds may lead to the formation of a mixture with superior properties compared to the simple physical mixtures of their components. Accordingly, a co-processed product as embodied herein comprises a mixture of the individual components or compounds, which results from the co-processing procedure, and which possesses unique characteristics and superior properties as compared to the simple physical mixtures of those components or compounds. For example, the co-processed nasal rinse powder may have enhanced dissolution, solubility, storage, and treatment properties and characteristics. One of the aims of co-processing is to obtain a product with added value related to the ratio of its functionality and price. The co-processed compounds may be selected and proportioned to get optimized product with desired physical and chemical parameters and it ends with minimizing avoidance with batch-to-batch variations. For example, a salt carrier may be combined with the optimal amount of a functional material, such as honey, in order to obtain integrated product, with superior functionality than the simple mixture of components.

Co-processing is valuable because the compounds of the resulting nasal rinse powder are physically modified in a special way without altering the chemical structure. In one embodiment, a fixed and homogenous distribution for the components may be achieved by embedding them within mini-granules. The components of the co-processed nasal rinse powder may be utilized to make use of the advantages of each component and to overcome specific disadvantages, such as the hygroscopic nature of dried honey.

Next, the system dries the carrier mixture (step 206). The carrier mixture may be dried utilizing any number of technologies capable of co-processing both honey and salt carrier to create a matrix composition. These include roll drying, fluid bed drying, thin film drying, freeze drying, vacuum drying, infrared drying, high or low temperature spray drying, spray congealing, or any such technique used to recover a dissolved solid by removal of water from a solution.

Next, the system mixes the powdered carrier mixture with additional ingredients to form the nasal rinse powder (step 208). The mixing may be performed in a powder blender such as a V-blender, ribbon blender, double-cone blender or any other type of blender used to blend multiple ingredients. In a preferred embodiment, the additional ingredients may be sodium chloride and/or sodium bicarbonate. Other examples of additional ingredients may include nasal probiotics, vitamins, herbs, extracts, oils, excipients, or other components. For example, the additional ingredients may include compounds or materials, such as 1-Argenine, turmeric, angelica, mint, chrysanthemum, magnolia, ipratropium bromide, kali bichromicum, triamcinolone acetonide, pulsatilla, diphenhydramine, chlorpheniramine, hydroxyzine, hepar sulph, azelastine, thuja, spigelia, fluticasone, flunisolide, garlic, horseradish, cod liver oil, Vitamin C, or menthol. In another aspect, the additional ingredients may include other compounds with antimicrobial activity, such as, for example, coconut oil, or derivatives of coconut oil, including aprylic acid, capric acid, and lauric acid. These naturally occurring compounds are known to possess antimicrobial and antifungal properties, and their inclusion could provide additional or complementary benefits to the nasal rinse powder compositions.

In one embodiment, the additional ingredients or components of the nasal rinse powder are added during step 208 to prevent deactivation or modification of materials that may be materially changed by the drying process of step 206. In another embodiment, the additional ingredients of the nasal rinse powder are added during step 208 to treat specific conditions related to allergies, sinusitis, or rhinitis.

In one embodiment, the particle size of the nasal rinse powder is 100% passing a standard US 20 mesh sieve and not more than 35% passing a standard US 200 mesh sieve.

In another embodiment, the nasal rinse powder may be allowed to absorb water from the air or mixed with the powder to form a gel or concentrate. The nasal rinse powder may also include any number of food-grade or medical-grade surfactants. For example, the nasal rinse power may include a wet or dried form of baby shampoo that act as a mucolytic for dissolving biofilms by reducing water surface tension including various classes of surfactants or anionics. Baby shampoo may also act as an antimicrobial agent. For example, the surfactant may include sodium trideceth sulfate. Any number of other components may be utilized to create a homogeneous gel or concentrate for application or future mixing.

Next, the system packages the nasal rinse powder (step 210). In one embodiment, the nasal rinse powder is added to packages, bottles, vials, or containers. In another embodiment, the nasal rinse powder may be combined with a liquid, such as a saline or water, and then packaged in a rinse or spray bottle. The system may then distribute the nasal rinse powder for utilization by a number of users. In one embodiment, each package of the nasal rinse powder is marked with the batch, honey specific region, manufacturing date, and other relevant information. In another embodiment, each package of the nasal rinse powder is marked with the packaging date and/or best used by date. It is understood that because of the enduring properties of honey and the various carriers, the nasal rinse powder is highly stable, and should remain viable for months or even years while still being effective.

Once received by a user (e.g. purchased or provided by a medical professional), a nasal rinse solution may be prepared by dissolving the nasal rinse powder in a liquid, for example water. The user may utilize distilled water, but purified or clean tap water may also be used. The use may alternatively use another liquid, such as, for example, saline solution or a pH balanced solution. The packets containing nasal rinse powder compositions, for example compositions comprising honey and a carrier, such as sodium chloride and/or sodium bicarbonate, are combined with water to prepare a pH balanced solution. The packets may be available in any number of sizes associated with the user or organizational needs.

In one embodiment, the composition further comprises naturally occurring or medicated ingredients to form a pH balanced, honey-based, saline solution that is compatible with the human nasal and sinus mucosa to prevent burning or stinging during nasal rinsing.

In one aspect, the nasal rinse powder composition may comprise a unique formulation of dried honey and salts for nasal irrigation. In one embodiment, the composition comprises a mixture of raw honey, sodium chloride and/or sodium bicarbonate or other suitable salt carriers. In one aspect, the mixture of raw honey, sodium chloride and/or sodium bicarbonate or other suitable salt carriers may be dissolved or dispersed in water. In another aspect, the mixture of mixture of raw honey, sodium chloride and/or sodium bicarbonate or other suitable salt carriers dissolved or dispersed in water may be subsequently co-processed, for example including by drying the composition to remove the water. The amount of solids, such as sodium chloride, sodium bicarbonate, sugars (i.e. in honey), or so forth, may be present at a range from 1% to 99%. In a more preferred embodiment, the solids are present at between 70% and 90%. In an even more preferred embodiment, the solids are present at about 82%. The amount of solid material present will depend on the drying technology utilized.

In one embodiment, a roll dryer is utilized to dry the liquid honey and carrier solution. However, drying may be performed in a spray dryer, thin film dryer, freeze dryer, roll dryer (drum dryer) or by some other suitable means.

The composition may range from 1% to 99% honey by dry weight. The composition may range from 1% to 99% salt, on a dried basis. In a preferred embodiment, the ratio of salt to honey may range from 1:2 to 5:1. In another preferred embodiment, the ratio of salt to honey may range from 2:3 to 3:2. In a more preferred embodiment, the ratio of dried salt to honey in the composition is approximately 1:1.

The moisture content of the dried salt/honey mixture may range from 0-30%. In a more preferred embodiment, the moisture content is about 10% moisture. The dried salt/honey mixture may be blended with additional salt and sodium bicarbonate. The levels of honey may range from 1% to 85%. In a preferred embodiment, the components may be blended to give a final composition of approximately 65% sodium chloride, 20% sodium bicarbonate, and 15% honey.

The nasal rinse powder or blend is packaged into individual packets for dilution with water. Packaging of the individual servings may be in stick packs, sachets, vials, bags, bottles, or some other suitable container.

In one embodiment, the nasal rinse powder is packaged in a stick pack that is easily opened and poured. The ideal packaging configuration is a stick pack. In another embodiment, the blend may be packaged directly into a bottle or canister for use by the consumer. The nasal rinse may also be created and stored in aqueous or liquid form.

In one embodiment, a small measuring spoon, measuring cup, or measuring lines along with labels or instructions are provided for accurate dilution. The blend may be diluted in water by the consumer. Distilled water is recommended. However, boiled water may be used as a substitute for distilled water.

In other embodiments, other components, including antioxidants, antibiotics, steroids, antifungals, microorganisms, or probiotics may be included in the nasal rinse power, gel, concentrate, or solution. In one aspect, the additional component or components may be selected among antibiotics (e.g., aminoglycosides, carbacephem, carbapenems, cephalosporins, glycopeptides, penicillins, polypeptides, quinolones, sulfonamides, tetracyclines and the like); antifungals (e.g., polyene antibiotics, imidazole and triazole, allylamines); intraocular pressure lowering agents (e.g., alpha-adrenergic agonists, beta-adrenergic blockers, carbonic anhydrase inhibitors, cannabinoids, derivatives and prodrugs); antiallergic compounds (e.g., olapatadine, ketotifen, azelastine, epinastine, emedastine, levocabastive, terfenadine, astemizole and loratadine); biological agents (e.g., antibodies or antibodies fragments, oligoaptamers, aptamers and gene fragments, oligonucleotides, plasmids, ribozymes, small interference RNA, nucleic acid fragments, peptides and antisense sequences); growth factors (e.g., epidermal growth factor, fibroblast growth factor, platelet derived growth factor, transforming growth factor beta, ciliary neurotrophic growth factor, glial derived neurotrophic factor, NGF, EP0 and P1GF); immunomodulating agents (e.g., glucocorticoids, drugs acting on immunophilins, interferons, opioids); cytostatics (e.g., alkylating agents, antimetabolites and cytotoxic antibiotics); antioxidants (e.g., alpha-tocopherol, ascorbic acid, retinoic acid, lutein and their derivatives, precursors or prodrugs; UV-filter compounds (e.g., benzophenones); anti-redness agents (e.g., naphazoline, tetrahydrozoline, ephedrine and phenylephrine); fatty acids (e.g., omega-3 fatty acids), and the like, and any combination thereof. For example, new nasal probiotic materials are being discovered daily and it is expected that these probiotics be included in the illustrative embodiments in a compatible format. In one embodiment, rosmarinic acid may be included in the nasal rinse powder because of its antibacterial and anti-inflammatory properties. In another embodiment, the nasal rinse powder may include 1-arginine. Tumeric or bitter orange oil may also be included in the composition for its anti-inflammatory properties. In a preferred embodiment, the nasal rinse powder many also include coconut oil, or derivatives of coconut oil, including aprylic acid, capric acid, and lauric acid. These naturally occurring compounds are known to possess antimicrobial and antifungal properties, and their inclusion could provide additional or complementary benefits to the nasal rinse powder compositions. N-acetyl cysteine may be utilized for helping flush and clear out mucus. Various nasal probiotics and normal flora organisms may be used included in the nasal rinse powder or liquid solution. For example, lactobacillus acidophilus, a probiotic, is known to provide a benefit for those suffering from chronic rhiosinusitus. Helminths may also be included in the nasal rinse powder or solution. Other microorganisms, such as trichuris suis, are being studied as having potential advantages.

In one embodiment, the combination levels may be as shown in Table 1:

TABLE 1
Formulation of an exemplary embodiment (% dry wt.)

| Ingredient | Minimum Level | Maximum Level |
|---|---|---|
| NaCl | 35% | 95% |
| NaHCO₃ | 2% | 25% |
| Honey | 4% | 45% |

In another embodiment, the nasal rinse powder may be dry-processed. First, the honey may be dried. For example, the honey may be dried utilizing infrared processing, drum or roller drying, spray drying, or other moisture removal systems. The honey may be dried alone or may be mixed with a component to facilitate drying, such as glucose. The dried honey components may be hygroscopic (water craving) even drawing water molecules from the air through absorption or adsorption, such as capillary condensation and other similar phenomenon. These properties may also help the nasal rinse powder dissolve in water more effectively. As a result, the honey may be dried and processed in a low moisture/humidity environment to maintain the powdered state of the honey.

Next, the dried honey may be mixed with sodium chloride and/or sodium bicarbonate. The dried honey may be dry blended or mixed utilizing any number of other techniques. For example, any number of blenders may be utilized including ribbon, V, continuous processor, cone screw, screw, double cone, double planetary, high viscosity, counter-rotating, double and triple shaft, high shear, dispersion, paddle jet, vibration, mobile, drum, and planetary blenders or mixers may be utilized.

Next, the nasal rinse powder is packaged or sealed in individual units or in bulk to prevent moisture from accumulating in the powder. The nasal rinse powder may be generated in a low-humidity environment to prevent damping, caking, clumping, and water acquisition by the compounds of the nasal rinse powder. The nasal rinse powder may utilize any number of vitamins, herbs, acidifiers, carbonates, salts, and honey types to provide a more effective, better smelling (and tasting), and desirable nasal rinse for users. In another embodiment, the nasal rinse powder may be bound to or include an agent that prevents water acquisition or that encourages water acquisition when mixed. The nasal rinse powder may be utilized for a large scale rinse (e.g. 8 ounce or more treatment) or an equally effective micro treatment of a few milliliters.

As is well known, the illustrative embodiments are not intended for infants and may even be harmful because their digestive systems are not mature enough to prevent bacterial spores in honey from forming toxins in their bodies. These toxins may allow bacteria to grow in the infant's intestines causing a condition known as infant botulism.

In another embodiment, the powder as herein described may be packaged as a seasoning or ingredient. For example, the powdered honey mixture may be utilized for cooking, camping, hiking, backpacking, emergency kits, or so forth. The amount of a salt-based or other carrier may be adapted to meet the specific taste or ingestion demands. The amount of carrier or composition may be varied from salt-based carriers to gelatin or starch based carriers for allowing dried honey to function as a sugar replacement. For example, the powdered honey may be utilized to baste breads or meats before, during, or after being prepared.

Honey may be used in the various embodiments because of its osmotic effect, hydrogen peroxide, acid, and honey methyglycoxal (MGO or mogo) properties and composition. For example, due to the high concentration of 2 different monosaccharides (sugars), and low water content in honey, water is drawn out of bacteria (e.g. dehydrating the bacteria) and making growing conditions difficult for bacteria in the presence of honey. An enzyme in honey (glucose oxidase) causes the slow generation of hydrogen peroxide from the sugars, with hydrogen peroxide being toxic to bacteria. The low pH (acidity) of honey naturally prevents growth of bacteria. The MGO in honey has antibacterial activity.

The benefits and utility of honey including the individual proteins, amino acids, DNA, and building blocks of honey continue to be discovered every day. In addition, the effectiveness of nasal irrigations is disclosed in documents, such as Diane G. Heatley, M.D., The Safety and Efficacy of Nasal Saline Irrigation, which includes a summary of 100 clinical studies, journal reviews, and vital statistic surveys demonstrating the efficacy of saline irrigation in treating or alleviating the symptoms associated with sinus disease.

The previous detailed description is of a small number of embodiments for implementing the invention and is not intended to be limiting in scope. The following claims set forth a number of the embodiments of the invention disclosed with greater particularity.

What is claimed:

1. A co-processed composition, comprising:
a dried particulate comprising honey and a carrier in which the ratio of the carrier to honey is 99:1 to 1:99.

2. The co-processed composition of claim 1, wherein said carrier is sodium chloride.

3. The co-processed composition of claim 1, wherein the dried particulate is a co-processed product produced from a solution comprising said carrier and said honey.

4. The co-processed composition of claim 1, further comprising sodium bicarbonate.

5. The co-processed composition of claim 1, wherein said honey is selected from the group consisting of: honey indigenous to the region where the product is being distributed, Manuka honey, and a biologically engineered or processed honey.

6. The co-processed composition of claim 1, wherein the carrier is potassium chloride.

7. The co-processed composition of claim 1, wherein said ratio of said carrier to said honey is 1:1.

8. The co-processed composition of claim 1, further comprising one or more additional components, selected from the group consisting of: antioxidants, antibiotics, steroids, antifungals, microorganisms, or probiotics.

9. A nasal rinse powder, comprising:
a dried honey particulate; and
a salt based constituent.

10. The nasal rinse powder of claim 9, wherein the dried honey particulate further comprises a carrier.

11. The nasal rinse powder of claim 10, wherein said carrier is dextrose or maltodextrose.

12. The nasal rinse powder of claim 9, wherein the salt based constituent comprises sodium chloride.

13. The nasal rinse powder of claim 9, wherein the salt based constituent includes at least sodium chloride and sodium bicarbonate.

14. The nasal rinse powder of claim 9, further comprising one or more additional components, selected from the group consisting of: antioxidants, antibiotics, steroids, antifungals, microorganisms, or probiotics.

15. A method for manufacturing a powdered nasal rinse, the method comprising:
combining honey in liquid form with a first carrier to form a carrier mixture;
drying the carrier mixture to produce a powdered carrier mixture; and
mixing the powdered carrier mixture with a second carrier to produce the powered nasal rinse.

16. The method according to claim 15, wherein said first carrier is sodium chloride.

17. The method according to claim 16, wherein the honey to sodium chloride ratio in the powdered nasal rinse is approximately 1 to 1.

18. The method according to claim 16, wherein the honey to sodium chloride ratio is between 2:3 and 3:2 honey to sodium chloride.

19. The method according to claim 15, wherein the second carrier is sodium bicarbonate.

20. The method according to claim 15, wherein the first carrier is NaCl, the second carrier is NaHCO₃, and wherein the powdered nasal rinse comprises approximately 65% NaCl, 20% NaHCO₃, and 15% honey by dry weight.

21. The method according to claim 15, wherein the powdered nasal rinse has a moisture content of approximately 10%.

22. A method for manufacturing a powdered nasal rinse, the method comprising:
combining honey in liquid form with one or more carriers to form a carrier mixture; and
drying the carrier mixture to form the powdered nasal rinse.

23. A method for treating or preventing a nasal infection, rhinitis, or sinusitis, comprising administering a nasal rinse solution comprising a nasal rinse powder dissolved in a physiologically acceptable liquid, wherein said nasal rinse powder comprises honey and a carrier.

24. A system for preparing a composition for the treatment or prevention of a nasal infection, rhinitis, or sinusitis comprising:
a nasal rinse powder comprising a dried honey particulate and a salt carrier; and
a container or packet;
wherein said nasal rinse powder is contained within said container or packet.

25. A nasal rinse comprising honey and a liquid, wherein said liquid is selected from the group consisting of water, distilled water, or saline.
